(19) United States
(12) Patent Application Publication
(10) Pub. No.: US 2010/0035352 A1
(43) Pub. Date: Feb. 11, 2010

(54) Method for Monitoring a Childbirth Process

(76) Inventors: Johan Itzel, Danderyd (SE); Eva Itzel, Danderyd (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 12/063,782

(22) PCT Filed: Oct. 9, 2006
(86) PCT No.: PCT/US06/39377

§ 371 Date: Apr. 30, 2009

Related U.S. Application Data
(60) Provisional application No. 60/596,673, filed on Oct. 12, 2005.

(51) Int. Cl. G01N 33/00

(52) U.S. Cl. 436/93

(57) ABSTRACT

A liquid carrying element, such as a test strip, is preferably tagged and used together with a measurement and presentation device. The measurement and presentation device is used in labor wards to evaluate change in lactate levels in amniotic fluid. In order to make sure that the correct test strip is inserted in the measurement device an identification mechanism may be used. The test strip may contain an identification tag. The measurement device may be set to only accept correctly tagged test strips.

### TECHNICAL FIELD

The present invention relates to a method for monitoring a childbirth process of a pregnant woman.

### BACKGROUND OF THE INVENTION

One problem in today's delivery methods is that women suffer from dystocya during labor. This could result in that the delivery does not progress as desired and that the labor is drawn out without a successful natural childbirth. The pregnant woman may become frustrated and it may be necessary to use methods such as, vacuum, forceps or caesarean to deliver the baby. The dystocya of the pregnant woman may also expose the fetus to injury and fatigue.

The lactate concentration in the blood of the fetus has been measured in the past to control that the fetus does not suffer from oxygen deficiency. However, the lactate concentration in the fetus does not indicate the condition of the pregnant woman. There is a need to more effectively determine and control the condition of woman suffering from dystocya at an early stage to avoid unnecessary labor before using surgical and alternative childbirth methods.

There is also a need for an effective apparatus to be able to determine the above-outlined conditions. In addition to the above needs, the apparatus should also handle at least cervix dilatation data, fetal positioning data and fetal lactate and pH measurements.

### SUMMARY OF THE INVENTION

The method of the present invention provides a solution to the above-outlined problems. More particularly, the method is for tagging disposable articles that are used together with an apparatus for monitoring a childbirth process of a pregnant woman. The use of an identification mechanism such as a tag makes sure that the liquid carrying elements that are used together with the apparatus have the correct production origin. The monitoring of the childbirth process is done by an apparatus that measures lactate concentrations and evaluates the levels to determine whether the lactate concentrations are changing. The apparatus may, among other things measure, enter and display the information from the respective methods and sensors.

Together with the apparatus a multitude of liquid carrying disposables may be used. In order to ensure that the measurements presented to the medical practitioner is correct it is important that the correct disposables are used in order to not give inaccurate information. The disposables of the present invention may therefor be marked with a tag or any other suitable identification method that can be detected by the apparatus.

### BRIEF DESCRIPTION OF THE DRAWING

FIG. 1 is a schematic flow chart showing some of the steps of the method of the present invention;

FIG. 2 is a schematic illustration of graphical representation of measured data;

FIG. 3 is a schematic illustration of a test strip and a measurement device;

FIG. 4 is an exploded perspective view of an alternative embodiment of the device of the present invention;

FIG. 5 is a perspective view of the device shown in FIG. 4; and

FIG. 6 is a perspective view of the device inserted in a display unit.

### DETAILED DESCRIPTION

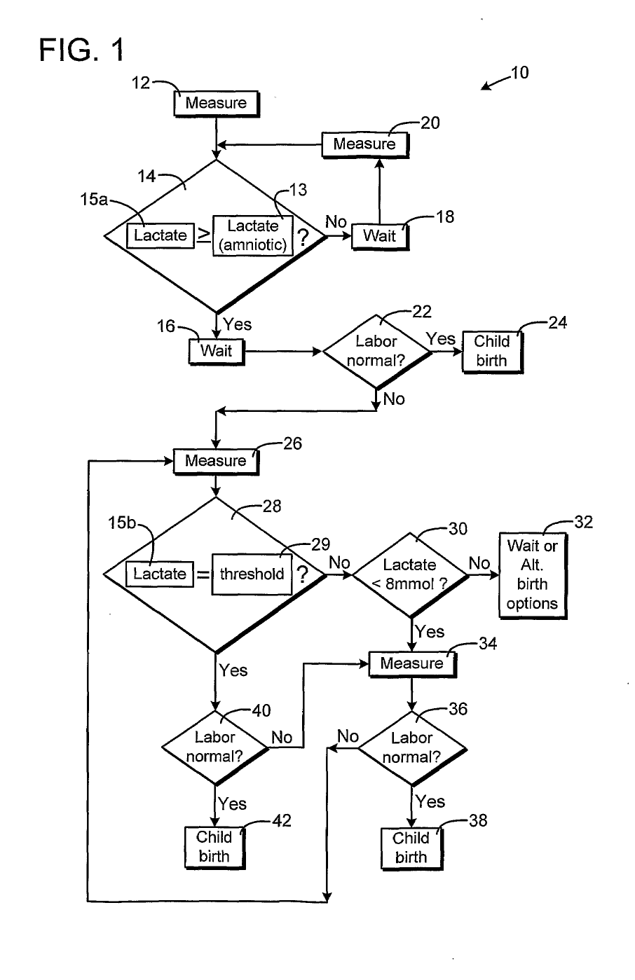

With reference to FIG. 1, the method 10 of the present invention includes a measuring step 12 that measures a lactate concentration 15a in fluids, such as vaginal fluids, in connection with pregnancy to determine whether the amniotic fluids have passed or are in the process of being passed from the amnion. In general, the uterus muscle of pregnant women produces lactate so that the lactate concentration of the vaginal fluids may be measured to provide a measurement of the amount of lactate produced by the uterus muscle. Non-pregnant women often have no or very little lactate in the vaginal fluids.

If the lactate concentration 15a is higher than a predetermined lactate concentration 13, such as 4-5 mmol/l, more preferably higher than 4.5 mmol/l, as indicated in a comparison step 14 then it may be concluded that the membrane has ruptured and amniotic fluids likely have passed and that the childbirth labor is likely to start after a waiting period 16. It is to be understood that the 4-5 mmol/l is an illustrative example that applies to most women and that the invention is not limited to the values used in the examples.

If the lactate concentration is lower than 4.5 mmol/l then there is a high likelihood that the amniotic fluids are still contained within the amnion. The lactate concentration may again be measured in a measuring step 20 after a waiting period 18. It is again determined in the comparison step 14 whether the lactate concentration is more or less than 4.5 mmol/l. If the lactate concentration is again below 4.5 mmol/l, a second measuring may be conducted later and the measuring may be repeated at suitable time intervals until the lactate concentration exceeds 4.5 mmol/l or it is obvious that the amniotic fluids have passed.

As indicated above, if the lactate concentration measured in the measuring step 12 is above 4.5 mmol/l, the next step is to wait for about two days or so to see if the woman starts the labor by herself. In a determining step 22, it is determined whether the labor has started or not. If the labor has started and is progressing normally then the childbirth procedure 24 may proceed. If it is determined in the determining step 22 that the labor has not started or the labor is not progressing normally, a lactate concentration 15b is measured in a measuring step 26.

In a comparison step 28 it is then determined if the lactate concentration 15b as measured in the measuring step 26 is within a lactate threshold interval 29 that may be about 8-10 mmol/l. If the lactate concentration as measured in the step 26 is not within the threshold interval 29, then it is determined in a comparison step 30 whether the lactate concentration is less than the threshold interval 29 or about 8 mmol/l. If the lactate concentration as measured in step 26 is greater than the threshold interval 29 then a waiting step 32, such as a couple of hours, may start to see if the labor progress normally. If labor does not progress normally, alternative childbirth options may be considered such as caesarean, forceps or the use of suction cups that are connected to vacuum to draw out the baby. An important feature of the present invention is that the monitoring of the lactate concentration may be used to predict whether the woman is likely to give a natural birth or not without forcing the pregnant woman to go through long and agonizing efforts to give birth. It is therefore possible to use alternative childbirth options at a relatively early stage. It is to be understood that the 8-10 mmol/l is an illustrative example that applies to most women and that the invention is not limited to the values used in the examples.

If the lactate concentration, as measured in step 26, is less than the threshold interval 29, then the woman may be stimulated with drugs or other aids to give birth in a stimulation step 34. In a determining step 36, it may be determined if the labor is progressing normally. If the labor is progressing normally the woman may proceed to give birth 38. If the labor is not progressing normally, the lactate concentration may again be measured in the measuring step 26 and the process continues in the comparison step 28, as described above.

If it is determined in the comparison step 28 that the lactate concentration, as measured in step 26, is at the threshold interval 29, such as between 8-10 mmol/l, then it is determined whether the labor is progressing normally in a determining step 40. If labor is progressing normally, the woman may proceed to give birth 42. If labor is not progressing normally, the woman may be stimulated to give birth in the stimulation step 34 and the process continues to the determining step 36, as described above.

The various processing loops may continue until the woman either gives birth by herself or is subjected to alternative childbirth options. As indicated above, an important feature of the present invention is that the woman may be prevented from agonizing and long childbirth efforts before alternative childbirth options are used. Alternative childbirth options may be used at an earlier stage when the lactate concentration indicates that the uterus muscle is operating above the lactate threshold without resulting in a natural childbirth.

Another important feature of the present invention is to use a measuring and presentation device to effectively present the measured lactate values together with the time lapsed. The depicted time intervals could be both on a relative time (time from start) or absolute time (time of day) to indicate the trends of the measured lactate values. In addition, the device may also be used to measure lactate levels in the fetus such as in the scalp blood of the fetus. The device may also be used to present the how far down in the pelvis the head of the fetus has penetrated and the dilation of the cervix.

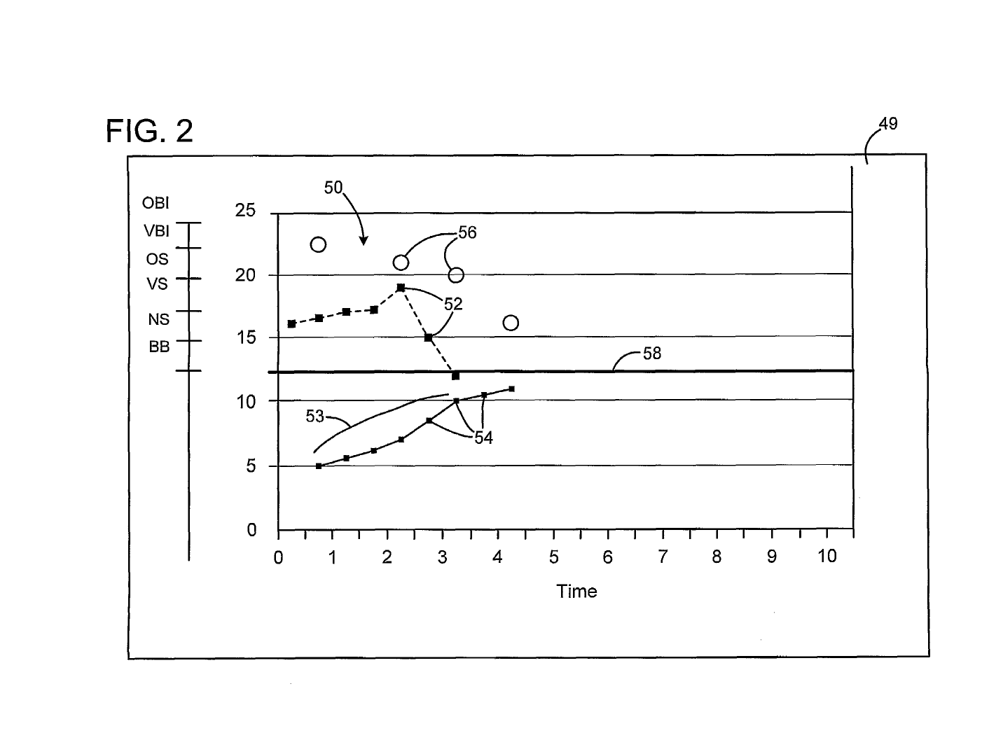

FIG. 2 is a display 50 is an illustrative example that shows lactate data 52 of the pregnant woman over time, lactate data 53 of the fetus, cervix dilation data 54 and fetal head positioning data 56. For example, the lactate data 52 shows an upward trend until the addition of stimulation substances to the pregnant woman is turned off and the lactate level is dramatically reduced. Because the display 50 shows that the lactate levels of the woman is already very high and rising, such as values above 15 mmol per liter, the medical practitioners may learn that there is no longer a need to provide stimulation to the pregnant woman. In this way, the display 50 may be used to prevent the misuse of stimulating substances. The medical practitioner may also use the trends indicated by the data 53, 54, 56 so that the practitioner can see all the data together and take all the information into account before making a decision about what action to take including medication required and the need for performing a caesarian or any other such aided child birth. For example, the cervix dilation data 54 may be compared to a normal cervix dilation curve 55 that applies to most women. The medical practitioner may also use the display 50 to see how the pregnant woman and fetus are reacting to treatment.

The practitioner may take a plurality of measurements of the lactate concentrations 52 at time intervals. The measured lactate concentrations 52 are then presented at the time intervals on the display 50. The lactate concentrations 52 are evaluated to determine whether the lactate concentrations 52 are rising to indicate that an individual lactate threshold value 58 of the pregnant woman has been exceeded. To be able to determine that the individual lactate threshold has been exceeded is particularly important for women who have an unusually high or low lactate threshold.

All the data 52, 53, 54, 56 and other data that have been displayed on the display 50 may be saved for later retrieval together with information of the time the data was measured. The data may be stored together with patient information. This data may be electronically transferred to remote equipment such as electronic medical records by using wireless or wired communication. The device may also be equipped with an automatic decision guiding and alarm system that is based on measured lactate values from mother, measured fetal lactate values, measured fetal pH values. The system may also base decisions and alarms on the opening size of cervix and the progression of the fetus head in the pelvis. Of course, a combination of the above information may be used. The devise 50 should be designed so that it can handle several childbirth processes at the same time in the same device. The stored data in the device may have the ability to enter and present the measured data in electronic medical records.

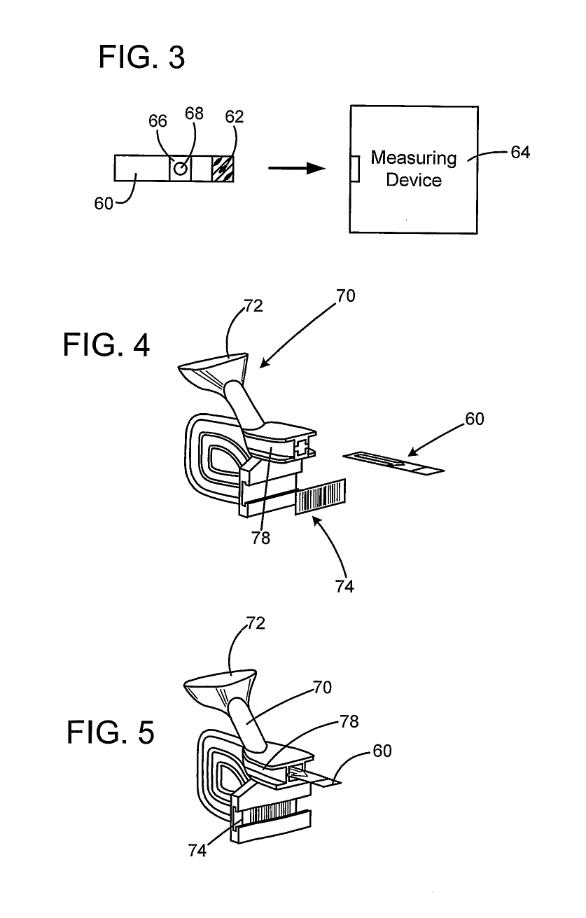

FIG. 3 shows a liquid carrying element such as a test strip 60 that is insertable into a measurement device 64. FIG. 3 is only an illustrative example of a liquid carrying element and the present invention is not limited to the test strip and the measurement device as shown. When a sample of liquid is collected for use with the measurement and presentation device the test strip or liquid carrying element 60 may be used to collect a liquid 62 from the pregnant woman that is to be analyzed. Preferably, the liquid to be analyzed is amniotic fluid or blood. The test strip may be inserted into a measurement and presentation device 64 either prior to or after the collection of the liquid. Preferably, the liquid 62 is deposited on the test strip 60. The device 64 then analyzes the liquid 62 such as determining a lactate concentration 15a, as described above. The test strips may be inserted in the measurement and presentation device as individual elements or as magazines containing several test strips. The measurement device 64 and the test strip 60 are both essential to make sure the data is accurate. The use of a non-conforming test strip can cause malfunction, or even worse, that invalid data is presented and, as a consequence, the pregnant woman is incorrectly treated.

Another important feature of the present invention is that in order to make sure that the correct test strip 60 is inserted in the measurement device 64 an identification mechanism 66 may be used on the test strip 60. The test strip may contain an identification tag 68. It is essential for correctness of the measured result that no contamination or other source of error is induced in the handling chain of the test sample of the liquid to be analyzed or measured. The tag 68 may therefore be designed so that the tag is detected by the measurement device 64 and give the measurement device the possibility of discriminating between correctly tagged test strips and untagged or unacceptable pirate strips. The tagging of the strips is a way to validate the origin of the test strips.

Also, other liquid carrying elements like tubes, catheters and bowls may be used in conjunction with the measurement device 64 and all such liquid carrying elements may also be tagged, as described above.

A wide variety of methods of tagging the strips or elements 60 may be used. For example, the tagging method may include a tuned antenna, resistive element on which the current/voltage is measured, mechanical key that can be detected, bar code that can be automatically scanned, radio frequency identification mechanisms or reflecting elements that can be optically read. The above mechanisms are only examples of how the mechanism of tagging in practice can be realized and the present invention of tagging the liquid carrying elements are not limited to the above examples.

The tag may apart from being used as a detection mechanism, also be used to validate the correct origin of the liquid carrying element and to carry other types of information. This information is supplementary and the absence of such does not mean that a tagging mechanism has not been implemented. The supplementary information that can be carried is for example, production date, calibration data, production batch information or any other suitable information.

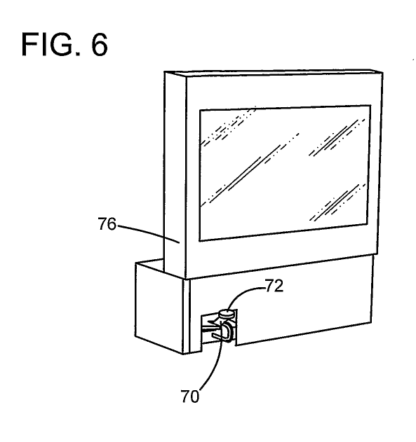

FIGS. 4-6 are perspective views of an alternative embodiment of the device of the present invention. The test strip 60 may be inserted into an other liquid carrying element 70 that has an inlet 72 defined therein for receiving liquid to be tested such as amniotic fluid discussed above. Identification tag or bar code 74 may be attached to the element 70 for identification purposes. FIG. 5 shows the element 70 completely assembled. The element 70 may then be inserted into and removably attached to a measurement device 76. The element 70 is removed therefrom when the analysis of the information has been displayed as a result of the information on the strip 60. The element 70 has guiding members 78 to ensure the element 70 is correctly inserted into the device 76. Once the element 70 (including the strip 60 and identification tag 74) is attached to the device 76, the liquid is poured into the inlet 72 and the strip 60 measures the content of the liquid. When the analysis is complete, the entire element 70 is removed from the device 76 and may be thrown away. The element 70 may contain a filter to ensure that foreign substances are removed from the measured samples While the present invention has been described in accordance with preferred compositions and embodiments, it is to be understood that certain substitutions and alterations may be made thereto without departing from the spirit and scope of the following claims.

1. A method of monitoring a childbirth process of a pregnant woman, comprising:
obtaining an amniotic fluid from the pregnant woman during labor;
carrying the amniotic fluid with a liquid carrying element;
determining a lactate concentration of the amniotic fluid carried by the liquid carrying element by inserting the liquid carrying element into a lactate measuring device; and
using the lactate concentration of the amniotic fluid to diagnose labor dystocia.

2. The method according to claim 1 wherein the method further comprises placing the amniotic fluid on a test strip.

3. The method according to claim 2 wherein the method further comprises inserting the test strip in a disposable casing.

4. The method according to claim 1 wherein the method further comprises tagging the liquid carrying element and setting the lactate measuring device to detect tagged liquid carrying elements.

5. The method according to claim 1 wherein the method further comprises the lactate measuring device measures a liquid carried by a catheter.

6. The method according to claim 1 wherein the method further comprises the lactate measuring device detecting an identification mechanism on the liquid carrying element prior to determining a lactate concentration of the amniotic fluid.

7. The method according to claim 1 wherein the method further comprises adding an identification mechanism on the liquid carrying element.

8. The method according to claim 1 wherein the method further comprises filtering the amniotic fluid disposed in the liquid carrying element to avoid foreign substances.

9. The method according to claim 1 wherein the method further comprises tagging the liquid carrying element by providing a bar code.

10. The method according to claim 1 wherein the method further comprises adding measurement calibration data to the liquid carrying element.
